(19) United States
(12) Patent Application Publication
(10) Pub. No.: US 2023/0065389 A1
(43) Pub. Date: Mar. 2, 2023

(54) Medical Puncture Device

(71) Applicant: Jeremy Madvin, Westlake Village, CA (US)

(72) Inventor: Jeremy Madvin, Westlake Village, CA (US)

(21) Appl. No.: 17/412,299

(22) Filed: Aug. 26, 2021

(51) Int. Cl. A61N 1/14; A61B 17/3211; A61H 39/08; A61M 5/32

(52) U.S. Cl. CPC A61N 1/14; A61B 17/3211; A61H 39/086; A61M 5/32

(57) ABSTRACT

The present invention relates to an advancement in a medical puncture device. The assembly is in the form of a skin and/or tissue puncturing device which includes a piercing member and a lower end with attached conductive wire designed to be inserted into a grounding port. This device passes the body's own excess voltage through the specific area being punctured simultaneous to the puncturing. It also simultaneously passes the earths ions through the same point of contact/penetration.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to a medical device and, in particular, to a device which relates to a medical puncture device.

### Description of the Related Art

Core components of alternative, lifestyle, and preventive medicine include nutrition, exercise, stress management, and relationships. Missing from this formula is the practice of Earthing (also commonly referred to as grounding; in this paper we will primarily use the term Earthing).

Earthing simply means reconnecting the conductive human body to the Earth's natural and subtle surface electric charge, an effortless lifestyle activity that systemically influences the basic bioelectrical function of the body. Doing so surprisingly stabilizes the physiology, reduces inflammation, pain, and stress, improves sleep, blood flow, and lymphatic/venous return to the heart, and produces greater well-being.

These are systemic, profound effects. People often report that after they start Earthing they feel and look healthier and younger. Those with pain report less pain.1 Even mood improves. Earthing is easy to do and often achieves rapid results, particularly for individuals with chronic health disorders.

There are two basic ways of Earthing: 1) indoors, involving inexpensive Earthing systems (conductive mats, pads, body bands, or patches) while sitting or sleeping, and 2), in its most natural form, and free, by spending time barefoot outdoors in contact with natural conductive surfaces, like grass, soil, gravel, stone, and sand.

Just as the production of vitamin D in our body is influenced by energy and frequencies generated from the sun, 93 million miles away, the Earth right under our feet provides unique energy and frequencies that directly influence our body.

Earthing restores a primordial electric connection to the Earth lost over time because of human lifestyle. Earthing appears to correct what we call an “electron deficiency syndrome,”3 an overlooked and likely significant cause of multiple health disorders.

It is believed that Earthing as a preventive/lifestyle strategy can counteract the sharp rise in non-communicable diseases throughout the world, a major challenge and barrier to global development. Such diseases include cardiovascular, respiratory, neurodegenerative, and auto-immune conditions, type 2 diabetes, and cancer. These disorders account for more than 40 million deaths annually, and affect all nationalities and socio-economic classes.

As is well known, the rise in global illness has brought with it a chronic pain epidemic engulfing perhaps as many as 20% of adults,6 and, as a further byproduct, an alarming spike in painkilling medication usage, side effects, and addiction. Because of this development the U.S. Government declared a health emergency in 2017.7 The problem is growing worldwide as well.

These are all conditions and issues we encounter in our clinical practices. Earthing offers a potent health and healing tool for prevention and therapy alike, a tool that can be used by itself or in conjunction with clinical treatments to address not just symptoms but root causes of symptoms and dysfunction that begin at the deep bioelectrical levels of the physiology.

Medical puncture devices are used in the medical field to puncture a patient's skin to obtain a capillary blood sample from the patient. In some diseases, such as diabetes, it is necessary to test the patient's blood regularly, for example to monitor the patient's blood glucose level. Furthermore, test kits such as cholesterol test kits often require a blood sample for analysis. Blood collection procedures generally involve puncturing a finger or an appropriate body part to obtain a blood sample. Usually, the amount of blood required for such a test is relatively small, and a small puncture or incision usually provides a sufficient amount of blood for such a test.

Various medical puncture devices are commercially available for hospitals, clinics, clinics, etc., as well as for personal consumers. Such devices typically use a pointed member, such as a needle, or a sharp-edged blade, that is used to quickly create a puncture or incision in the patient's skin for the purpose of producing a small amount of blood outflow. For many people, it is often physiologically and psychologically difficult to puncture their fingers with a hand-held needle or blade. As a result, medical puncture devices have evolved into automated devices that puncture or incise the patient's skin when the trigger mechanism is activated. In some devices, the needle or blade remains in a waiting position until triggered by the user, which may be a medical professional responsible for drawing blood from the patient or the patient himself. When triggered, the needle or blade punctures or incises the patient's skin, such as a finger. Often, a spring is incorporated into the device to provide the “automatic” force necessary to puncture or incise the patient's skin.

There are multiple solutions that have been presented in prior art. However, the present disclosure provides an advanced way of incorporating a grounding system with a medical puncturing device with the following claimed benefits. During the process of grounding, the bodies excess voltage escapes the body through the grounding device itself, stimulating the tissue the voltage passes through. In the case of acupuncture, this may stimulate the meridian point with the bodies own energy or the ions being transferred from the earth.

Furthermore, in the case of a hypodermic needle or a blood collection puncturing device, mild electrical stimulation has been demonstrated to cause a simultaneous, and prolonged dilation to the capillaries leading to better localized blood flow and better collection volume. Also, in the case of the scalpel, reducing the body's excess electrical charge will reduce the stress on the cells and possibly lead to speedier recovery among other benefits. This device is similar to an acupressure device however the acupuncture wire connects to a thin needle which penetrates the skin for acupuncture and other grounded invasive medical requirements.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

in light of the disadvantages of the prior art, the following summary is provided to facilitate an understanding of some of the innovative features unique to the present invention and is not intended to be a full description. A full appreciation of the various aspects of the invention can be gained by, taking the entire specification, claims, drawings, and abstract as a whole.

The present invention seeks to improve upon prior medical puncture devices and provide an enhanced design to perform the designated functionality.

Another object of the present invention is to provide, a medical puncturing device with a wire which connects to a thin needle which penetrates the skin for acupuncture, a hypodermic needle for injections, or in some other way to a scalpel for making incisions and other grounded invasive medical tools.

It is a further objective of the present invention to provide an improved structure which provides the traditional functionality of acupressure along with medical puncture device.

It is also the objective of the invention to provide an associated application which increases control and ease of use and also enhances both the speed and quality of the entire assembly.

It is also the objective of the invention to provide a whole construction which is compatible with high volume manufacturing methods for all of the components.

Thus, it is the objective to provide a new and improved form of an advancement of medical puncture devices. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates the perspective use of proposed assembly, in accordance with an embodiment of the present disclosure.

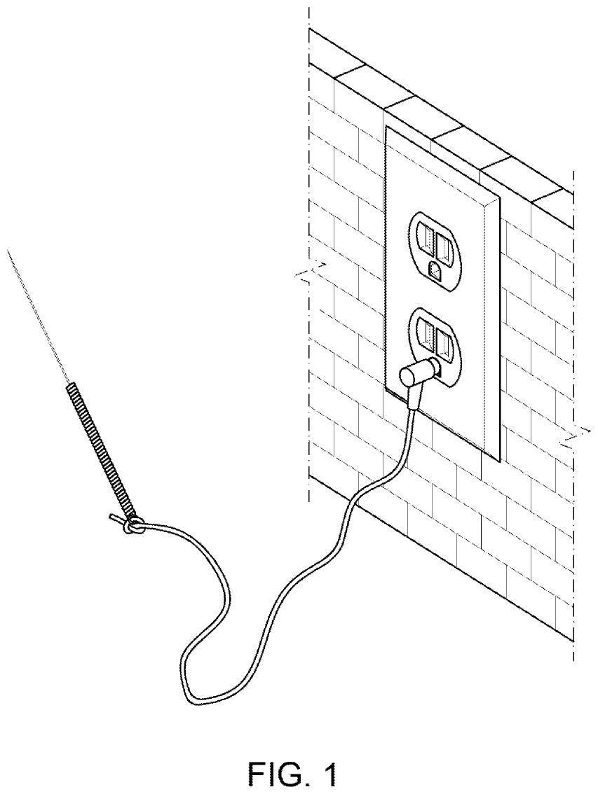

FIG. 2 illustrates another use of medical puncture device and view of the utilization of present invention, in accordance with an embodiment of the present disclosure.

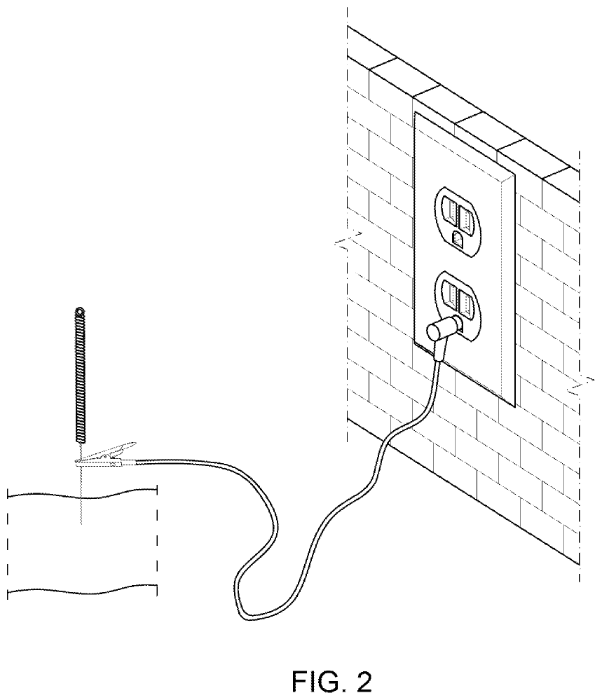

FIG. 3 illustrates another view of proposed assembly, in accordance with an embodiment of the present disclosure.

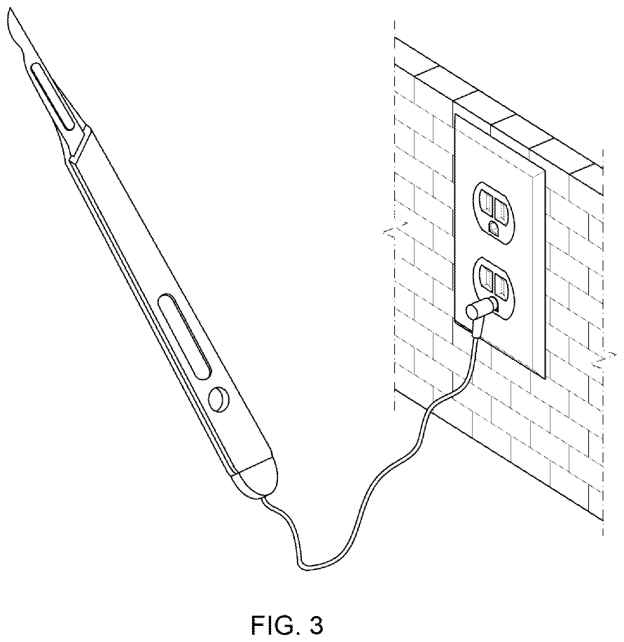

FIG. 4 illustrates front view of assembly, in accordance with an embodiment of the present disclosure.

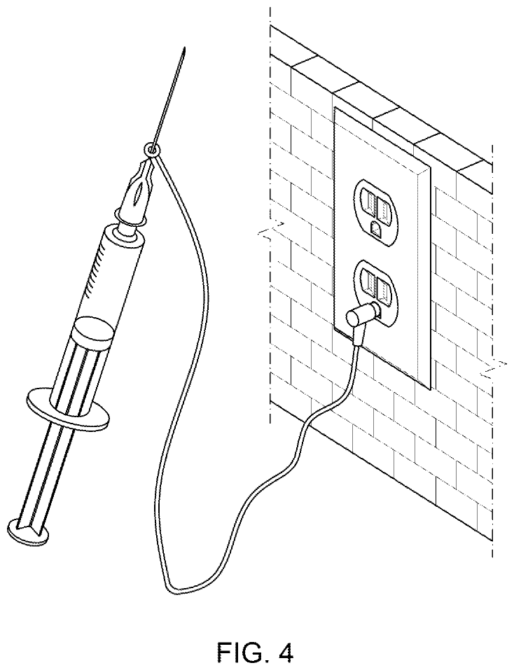

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In one aspect of the present invention, various medical puncture devices are commercially available for hospitals, clinics, clinics, etc., as well as for personal consumers. Such devices typically use a pointed member, such as a needle, or a sharp-edged blade, which is used to quickly create a puncture or incision in the patient's skin for the purpose of producing a small amount of blood outflow. For many people, it is often physiologically and psychologically difficult to puncture their fingers with a hand-held needle or blade. As a result, some medical puncture devices have evolved into automated devices that puncture or incise the patient's skin when the trigger mechanism is activated. In some devices, the needle or blade remains in a waiting position until triggered by the user, which may be a medical professional responsible for drawing blood from the patient or the patient himself. When triggered, the needle or blade punctures or incises the patient's skin, such as a finger. Often, a spring is incorporated into the device to provide the “automatic” force necessary to puncture or incise the patient's skin.

In recent years, safety has been improved in the operation and handling of medical puncture devices after use. For example, medical puncture devices are currently available that are single firing devices featuring automatic protrusion and retraction of the puncture or incision element from and into the device.

The current invention as per its preferred embodiments discloses a grounding wire which connects to a thin needle which penetrates the skin for acupuncture, a hypodermic needle for injections, a scalpel for making incisions and other grounded invasive medical tools.

The assembly as per its preferred embodiments discloses the grounding wire attaching to a scalpel, acupuncture needle and hypodermic needle is the part that connects the wire to each. For example, it could be a noose for the needle and an alligator clip for the scalpel

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

I: A grounding device comprising:
an inserter device for inserting a piercing member into the intramuscular region of a patient, where it passes the body's own excess voltage through the specific area being punctured simultaneous to the puncturing. It also simultaneously passes the earths ions through the same point of contact/penetration. The insertion device includes an upper part and a lower part. The puncture member protrudes from the housing to the aligned second position, and the grounding wire is attached to a scalpel.

II: A medical puncture device where the acupuncture wire connects to a thin needle which penetrates the skin for acupuncture, a hypodermic needle for injections, a scalpel for making incisions and other grounded invasive medical tools.
